(19) United States
(12) Patent Application Publication
(10) Pub. No.: US 2021/0280279 A1
(43) Pub. Date: Sep. 9, 2021

(54) GENERATING MEDICAL ANALYSIS USING A JOINT MODEL BASED ON MULTIVARIATE ORDINAL DATA

(71) Applicant: Otsuka America Pharmaceutical, Inc., Rockville, MD (US)

(72) Inventors: Srikanth Gottipati, Princeton Junction, NJ (US); Guoqing Diao, Chantilly, VA (US); Peter Zhang, Rockville, MD (US)

(21) Appl. No.: 17/194,188

(22) Filed: Mar. 5, 2021

Related U.S. Application Data
(60) Provisional application No. 62/985,832, filed on Mar. 5, 2020.

(51) Int. Cl. G16H 10/20; G16H 50/20; G16H 20/10; G16H 70/40

(52) U.S. Cl. CPC G16H 10/20; G16H 70/40; G16H 20/10; G16H 50/20

(57) ABSTRACT

Methods, systems, and apparatuses, including computer programs for analyzing multi-variate ordinal outcomes. In one aspect, the method can include actions of obtaining one or more answers to one or more questions of a first assessment, generating a first group of processing data based on the one or more answers, wherein the first group of processing data comprises one or more ordinal values, one or more covariates, and one or more factors, generating output data based on the first group of processing data, generating a medical analysis corresponding to the one or more answers provided by a first user based on the output data and one or more other outputs from one or more other generated groups of processing data, and sending the medical analysis to a first device.

### CROSS-REFERENCE TO RELATED APPLICATION

This application claims the benefit of U.S. Provisional Patent Application No. 62/985,832 filed Mar. 5, 2020, the entire contents of which is incorporated herein by reference in its entirety.

### BACKGROUND

Assessments may be used by professionals to obtain data from a user. Within the medical profession, assessments may be used to obtain data on the well-being of a patient.

### SUMMARY

Aspects of the present disclosure are directed towards assessment analysis. The assessment analysis may include generating a joint model of factor and regression analysis stages.

According to one innovative aspect of the present disclosure, a computer-implemented method for analyzing multi-variate ordinal outcomes is disclosed. In one aspect, the method can include actions of obtaining, by one or more computers, one or more answers to one or more questions of a first assessment, generating, by the one or more computers, a first group of processing data based on the one or more answers, wherein the first group of processing data comprises one or more ordinal values, one or more covariates, and one or more factors, generating, by the one or more computers, output data based on the first group of processing data, generating, by the one or more computers, a medical analysis corresponding to the one or more answers provided by a first user based on the output data and one or more other outputs from one or more other generated groups of processing data, and sending, by the one or more computers, the medical analysis to a first device.

Other versions include corresponding systems, apparatus, and computer programs to perform the actions of methods defined by instructions encoded on computer readable storage devices.

These and other versions may optionally include one or more of the following features. For instance, in some implementations, the method can further include receiving, by the one or more computers, a processing request from a second device, responsive to receiving the processing request from the second device, obtaining, by the one or more computers, the first assessment, wherein the first assessment comprises the one or more questions, wherein the first assessment is stored within an assessment database of one or more assessments, and wherein the assessment database is communicably connected to the one or more computers, and sending, by the one or more computers, the first assessment to the second device, wherein the second device enables the first user to provide the one or more answers to the one or more questions of the first assessment.

In some implementations, generating the first group of processing data can include extracting, by the one or more computers, the one or more ordinal values from the one or more answers, identifying, by the one or more computers, the one or more covariates from the one or more answers, and generating, by the one or more computers, the one or more factors based on the one or more answers.

In some implementations, the one or more factors can include one or more of negative symptoms, positive symptoms, disorganized thought, uncontrolled hostility or excitement, or anxiety or depression.

In some implementations, the one or more covariates can include one or more of treatment group, age, or gender.

In some implementations, the medical analysis comprises a determined effect of a drug treatment, wherein the first user is engaged in the drug treatment.

The details of one or more embodiments of the invention are set forth in the accompanying drawings and the description below. Other features and advantages of the invention will become apparent from the description, the drawings, and the claims.

### BRIEF DESCRIPTION OF THE DRAWINGS

FIG. 1 is a contextual diagram for an example of a system for generating medical analysis using a joint model based on multivariate ordinal data.

FIG. 2 is a flowchart for an example of a process for generating medical analysis using a joint model based on multivariate ordinal data.

FIG. 3 is a flowchart for an example of a process for establishing a psychiatric assessment and generating medical analysis using a joint model based on multivariate ordinal data.

FIG. 4 is a diagram of computer system components that can be used to implement a system for generating medical analysis using a joint model based on multivariate ordinal data.

Like reference numbers and designations in the various drawings indicate like elements.

### DETAILED DESCRIPTION

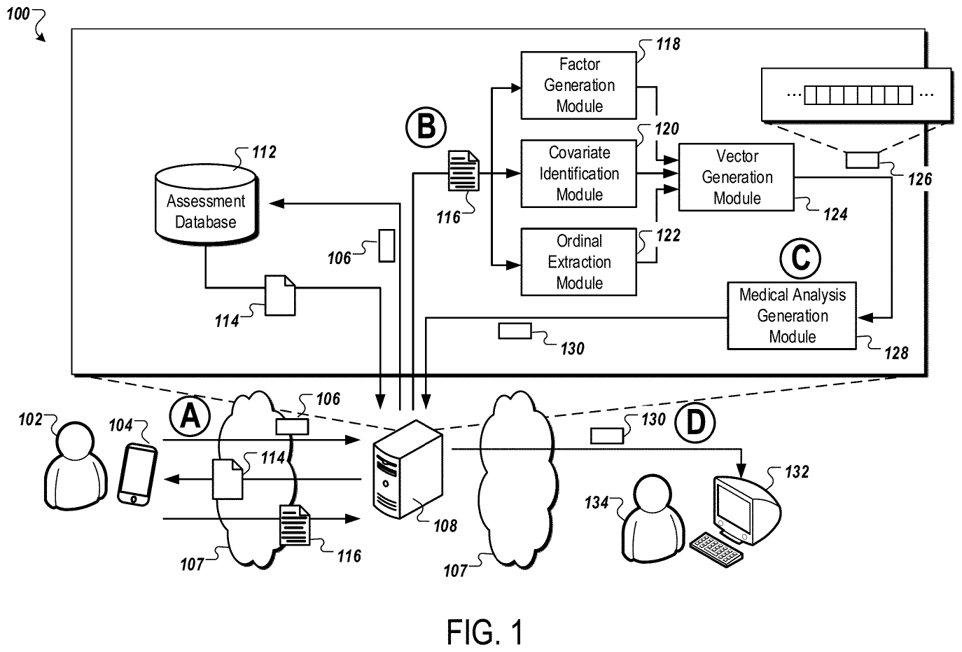

FIG. 1 is a diagram showing an example of a system 100 for generating medical analysis using a joint model based on multivariate ordinal data. The system 100 can include a communication network 107, a first computer 108, an assessment database 112, a factor generation module 118, a covariate identification module 120, an ordinal extraction module 122, a vector generation module 124, and a medical analysis generation module 128. The first computer 108 is communicably connected to the first user device 104 and the second user device 132 by the communication network 107. The assessment database 112, the factor generation module 118, the covariate identification module 120, the ordinal extraction module 122, the vector generation module 124, and the medical analysis generation module 128 are either stored on the first computer 108 or stored on one or more devices communicably connected to the first computer 108. Processors corresponding to the first computer 108 or the one or more devices communicably connected to the first computer 108 can perform operations attributed to the first computer 108, the factor generation module 118, the covariate identification module 120, the ordinal extraction module 122, the vector generation module 124, or the medical analysis generation module 128. The assessment database 112 may be stored within memory of the first computer 108 or within memory of the one or more devices communicably connected to the first computer 108. The example of FIG. 1 is discussed in reference to stages A through D.

The first user device 104 can include any client-side device used by a first user 102 to access one or more measurements or measurement-based tools communicably connected to the first computer 108. In some implementations, the first user device 104 can include a smartphone or a tablet device. In other implementations, the first user device 104 can include a smartwatch, a laptop computer, a desktop computer, or the like. The first user device 104 can include a variety of input and output devices, as known in the art. By way of example, the user device can include a camera 104, a speaker, a display unit, and a microphone that enable input data to be captured from the first user 102 and output data to be communicated to the first user 102.

The second user device 132 can include any device used by a second user 134 to access analysis data provided by the first computer 108. In some implementations, the second user device 132, similar to the first user device 104, can include a smartphone or a tablet device. In other implementations, the second user device 132 can include a smartwatch, a laptop computer, a desktop computer, or the like. The second user device 132 can include a variety of input and output devices, as known in the art.

In stage A of FIG. 1, the first user 102 uses the first user device 104 to send an assessment request 106 to the first computer 108. The assessment request 106 includes details related to the first user 102 and one or more assessments relevant to the first user 102. The assessment request 106 is sent over the communication network 107 to the first computer 108. The first computer 108 receives the assessment request 106 and retrieves a corresponding assessment 114, based on the input of the assessment request 106, from the assessment database 112. In some cases, the assessment database 112 stores one or more assessments. The first computer 108 obtains the assessment 114 from the one or more assessments stored on the assessment database 112. The assessment 114 is then sent by the first computer 108 to the first user device 104 over the communication network 107.

Once received by the first user device 104, the first user device 104 can display one or more items of the assessment 114 to the first user 102. In some cases, the one or more items of the assessment 114 may include a question related to assessing psychiatric disorders based on the Positive and Negative Syndrome Scale (PANSS) outcome instrument. The first user 102 may then provide answers to one or more questions included in the assessment 114. The first user device 104 may then generate a completed assessment 116 based on the assessment 114 and the answers provided to at least one question of the one or more questions included in the assessment 114. The first user device 104 sends the completed assessment 116 to the first computer 108 over the communication network 107.

In some implementations, another user may interact with the first user device 104 and interact with the first user 102 to obtain one or more answers. For example, a trained physician can interact with the first user device 104. The trained physician can interview the first user 102. Based on the interview, the trained physician can obtain answers to one or more questions of the assessment 114. The trained physician can then provide the answers to the one or more questions of the assessment 114 or provide one or more determinations based on the answers to the one or more questions of the assessment 114, to the first user device 104. Based on the answers to the one or more questions of the assessment 114 or based on the one or more determinations of the trained physician based on the answers to the one or more questions of the assessment 114, the first user device 104 can generate the completed assessment 116.

In some implementations, the completed assessment 116 is completed based on PANSS. For example, the first user 102 can be assessed in a clinical interview based on the assessment 114. The first user 102 can be rated on a numerical scale e.g., 1 to 7, based on one or more items, e.g., 30 items. The one or more items may include delusions, conceptual disorganization, hallucinations, excitement, grandiosity, suspiciousness/persecution, hostility, blunted affect, emotional withdrawal, poor rapport, passive/apathetic social withdrawal, difficulty in abstract thinking, lack of spontaneity and flow of conversation, stereotyped thinking, somatic concern, anxiety, guilt feelings, tension, mannerisms and posturing, depression, motor retardation, uncooperativeness, unusual thought content, disorientation, poor attention, lack of judgement and insight, disturbance of volition, poor impulse control, preoccupation, or active social avoidance. The first user 102, based on the assessment 114, may provide answers corresponding to 30 items included in a traditional PANSS score. The answers, or determinations based on the answers, may be included in the completed assessment 116.

In some implementations, the assessment request 106 includes information corresponding to the first user 102. For example, the assessment request 106 can include a name, ‘John Smith’, of the first user 102. The assessment request 106 can also include other information such as occupation, ethnicity, age, drug trial identification, diagnostic information, medical information, or the like. In some cases, the first computer 108 can include information from the assessment request 106 into the assessment 114 such that the first user 102 does not need to re-enter information. In some cases, the assessment request 106 is generated based on stored information corresponding to the first user 102. In some cases, the stored information corresponding to the first user 102 is stored on the first user device 104, a computer communicably connected to the first user device 104, or a database communicably connected to the first user device 104. As a result, the assessment request 106 can be generated by the first user 102 without re-entering information based on pre-entered data corresponding to the first user 102.

In some implementations, the completed assessment 116 includes one or more fields related to the assessment 114, the first user 102, or the first user device 104. For example, the completed assessment 116 can include a data structure that describes responses submitted by the first user 102 or determinations of a trained physician based on answers provided by the first user 102. The completed assessment 116 also includes information related to the first user 102. For example, the completed assessment 116 may include demographic information, name, age, preferences, a corresponding trial in which the first user 102 is enrolled, or the like. In some cases, the assessment 114 may be generated with information corresponding to the first user 102 based on the assessment request 106. In some cases, the completed assessment 116 includes a unique identifier.

In stage B, the first computer 108 receives the completed assessment 116. The completed assessment 116, based on information corresponding to the completed assessment 116 or a unique identifier of the completed assessment 116, is sent to various processing modules including the factor generation module 118, the covariate identification module 120, the ordinal extraction module 122, the vector generation module 124, and the medical analysis generation module 128.

The factor generation module 118 obtains, based on the completed assessment 116, one or more factors related to the completed assessment 116. For example, in the example of PANSS scores used for psychiatric patients, the one or more factors can include positive symptoms, negative symptoms, disorganized thinking, and the associated symptom domains of hostility/excitement, and depression/anxiety. Traditionally, the one or more factors can be used to interpret PANSS scores.

The covariate identification module 120 obtains, based on the completed assessment 116, one or more covariates related to the completed assessment 116. For example, covariates including age, gender, and race may be obtained by the covariate identification module 120. The effects of the covariates can then be evaluated based on factors obtained by the factor generation module 118, such as positive symptoms, negative symptoms, disorganized thinking, and the associated symptom domains of hostility/excitement, and depression/anxiety.

In some implementations, the covariate identification module 120 may obtain a threshold corresponding to the one or more covariates. For example, the covariate identification module 120 can obtain the one or more covariates or factors used as covariates using a Kaiser-Guttman criterion. The Kaiser-Guttman criterion can include limitations in which the covariate identification module 120 extracts as many covariates as there are sample eigenvalues greater than 1 within data corresponding to the completed assessment 116. In some implementations, the covariate identification module 120 may process the completed assessment 116 to generate latent factors. In some implementations, latent factors may include the one or more covariates obtained by the covariate identification module 120. For example, the covariate identification module 120 can parse data or process an image corresponding to the completed assessment 116. In some implementations, the covariate identification module 120 may obtain a pre-processed version of the completed assessment 116. For example, another connected component of the system 100 may process the completed assessment 116 and transmit data corresponding to the completed assessment 116 to the covariate identification module 120.

In some implementations, the number of covariates may be estimated through a module processing data corresponding to the completed assessment 116. For example, the covariate identification module 120 can obtain multivariate data, such as multivariate data parsed from the completed assessment 116. The multivariate data can be represented as a matrix of size n×q. In some implementations, the covariate identification module 120 may perform segmentation on the input data, such as the multivariate data. For example, the covariate identification module 120 can split the multivariate data into separate groups to improve subsequent processing. For each covariate, processing can be performed to (i) estimate a first matrix, such as a fitted correlation matrix, with a factor analytic structure based on a first group of covariates, (ii) estimate a second matrix, such as a ploychoric correlation matrix, based on a second group of covariates, and (iii) generate a goodness of fit measure based at least on the first matrix and the second matrix. In some implementations, the first group of covariates may be separate and distinct from the second group of covariates. In some implementations, based on the processing for each covariate, the covariate identification module 120 or a module communicable connected to the covariate identification module 120, can generate a threshold number of covariates to parse from the completed assessment 116. For example, the threshold number of covariates to parse from the completed assessment 116 can be a minimum corresponding to the goodness of fit measurements generated for each covariate, such as the minimum of a summation of the goodness of fit measurements.

In some implementations, a component of the system 100 may estimate the number of covariates without splitting input data corresponding to multivariate data. For example, the covariate identification module 120 can use various criterions, such as the Akaike information criterion or the Bayesian information criterion, among others, to determine a number of optimal covariates. The number of optimal covariates can then be extracted from input data such as data corresponding to the completed assessment 116 or the completed assessment 116 itself. In some implementations, criterion calculations may be minimized in order to generate an optimal number of covariates. For example, a model of the covariate identification module 120 can be trained or programed to find a suitable minimum corresponding to known functions of criterions such as the Akaike information criterion or the Bayesian information criterion.

The ordinal extraction module 122 obtains, based on the completed assessment 116, one or more ordinal values related to the completed assessment 116. For example, ordinal values related to one or more items corresponding to delusions, conceptual disorganization, hallucinations, excitement, grandiosity, suspiciousness/persecution, hostility, blunted affect, emotional withdrawal, poor rapport, passive/apathetic social withdrawal, difficulty in abstract thinking, lack of spontaneity and flow of conversation, stereotyped thinking, somatic concern, anxiety, guilt feelings, tension, mannerisms and posturing, depression, motor retardation, uncooperativeness, unusual thought content, disorientation, poor attention, lack of judgement and insight, disturbance of volition, poor impulse control, preoccupation, or active social avoidance may be extracted from the completed assessment 116. The scores corresponding to each item of the completed assessment 116 may be correlated with the factors obtained by the factor generation module 118. For example, the scores corresponding to each item may be grouped into factors such as positive symptoms, negative symptoms, disorganized thinking, and the associated symptom domains of hostility/excitement, and depression/anxiety.

In some implementations, the ordinal extraction module 122 may first obtain the one or more ordinal values based on the completed assessment 116. For example, the ordinal extraction module 122 can use image processing or digital parsing to determine the one or more ordinal values of the completed assessment 116. In some implementations, an image of the completed assessment 116 may be uploaded to the first computer 108. The image of the completed assessment 116 may then be processed by the ordinal extraction module 122 or a module communicably connected to the ordinal extraction module 122. Image analysis can be used to determine item numbers within the completed assessment 116 and corresponding answers for the one or more items of the completed assessment 116.

In some implementations, the completed assessment 116 may be sent to the first computer 108 as one or more data packets. For example, the one or more data packets can be configured to enable parsing by the first computer 108 or entity of the system 100, such as the ordinal extraction module 122. For example, the ordinal extraction module 122 can receive the one or more data packets of the completed assessment 116 and parse the one or more data packets to obtain the one or more ordinal values. In some implementations, the parsing of the one or more data packets may be based on one or more delimiting characters of the one or more data packets.

The factor generation module 118, the covariate identification module 120, and the ordinal extraction module 122 send corresponding data to the vector generation module 124. The vector generation module 124 generates vector data 126 based on the completed assessment 116 and the output of the covariate identification module 120, and the ordinal extraction module 122. The vector data 126 includes factors obtained by the factor generation module 118, covariates obtained by the covariate identification module 120, and ordinal values obtained by the ordinal extraction module 122.

In some implementations, factors may be obtained by the system 100 before receiving the completed assessment 116. In some implementations, factors may be obtained by the system 100 after receiving the completed assessment 116. In one example, factors may be generated based on a corpus of multivariate data related to one or more assessments. The corpus of multivariate data may be processed in order to extract one or more factors to be applied to subsequent assessments of the same or similar type. In another example, factors can be generated based on the system obtaining the completed assessment 116 and processing the completed assessment 116. Factors can be determined based on one or more factor loading matrices based on data from the completed assessment 116 or other similar or identical assessments.

In some implementations, the factor loading matrices are generated using a machine learning approach. For example, one or more elements of a factor loading matrix can be an output or an element of a machine learning model trained on one or more data groups corresponding to one or more assessments. Ground truth data may be used in order to help train one or more machine learning models based on one or more professional opinions or one or more follow up assessments to determine a true diagnosis of a user, such as the user 102. The one or more machine learning models can be used to provide data for any one of the components in the system 100 including the first computer 108, the factor generation module 118, the covariate identification module 120, the ordinal extraction module 122, the vector generation module 124, and the medical analysis generation module 128. In some implementations, each module is a distinct machine learning model trained to perform actions corresponding to the module as described herein. In some implementations, two or more of the modules are performed collectively using a single machine learning model in order to more efficiently process data corresponding to one or more assessments.

In stage C, the medication analysis generation module 128 receives the vector data 126 that includes factors obtained by the factor generation module 118, covariates obtained by the covariate identification module 120, and ordinal values obtained by the ordinal extraction module 122.

The medication analysis generation module 128 determines, based on the vector data 126, a joint model. The joint model is constructed based on elements of the vector data 126 including factors obtained by the factor generation module 118, covariates obtained by the covariate identification module 120, and ordinal values obtained by the ordinal extraction module 122. For example, suppose that y (Y₁, . . . , Y_{q}) where y is a q-dimensional vector of ordinal values obtained by the ordinal extraction module 122 and x is a set of p covariates obtained by the covariate identification module 120. Values of y can be defined by the following equation:

$${Y}_{j} = \{ \begin{matrix}1 , & {α}_{{j}_{0}} < {Y}_{j}^{*} \leq {α}_{{j}_{1}} \\ 2 , & {α}_{{j}_{1}} < {Y}_{j}^{*} \leq {α}_{{j}_{2}} \\ ⋮ & ⋮ \\ {K}_{j} & {α}_{{j}_{{K}_{j} - 1}} < {Y}_{j}^{*} \leq {α}_{{j}_{{K}_{j}}}\end{matrix} \qquad ( 1 )$$

In this example, Kⱼ represents categories for the jth ordinal outcome Yⱼ. The values of

$${α}_{{j}_{0}} , {α}_{{j}_{1}} , {α}_{{j}_{2}} … , {α}_{{j}_{{K}_{j}}}$$

correspond to

$$- ∞ = {α}_{{j}_{0}} < {α}_{{j}_{1}} < … < {α}_{{j}_{{K}_{j} - 1}} < {α}_{{j}_{{K}_{j}}} = ∞ .$$

The joint model of the medication analysis generation module 128 includes both factor and regression analyses of multivariate data. For example, the joint model may be represented by:

y*=Λξ+δ and ξ=B′x+ϵ (2)

The portion y*=Λξ+δ of Equation 2 may be generally described as one of multiple potential exploratory factor analysis (EFA) models that may be used in training or implemented by the system 100 of FIG. 1. In reference to the EFA model of Equation 2, the correlation structure of the underlying variable y* has the factorization Σ=ΛΦΛ′+Ψ where Λ is a q×k matrix of factor loadings, Φ is an identity matrix, and Ψ can be defined by an expression, such as I_{q}−diag(ΛΦΛ′).

In this example, y*≡(Y₁*, . . . , Y*_{q})′ and y* is a q×1 vector of underlying variables that determine the levels of the observed ordinal variables y≡(Y₁, . . . , Y_{q}). ξ is a k×1 vector of common factors. In some implementations, common factors are a form of latent variable. Λ is a q×k matrix of factor loadings λᵢⱼ where the factor loadings λᵢⱼ applied to factor ξᵢ are added to δ, where δ, is a q×1 vector of residuals in a factor model, to generate each value Yᵢ* of y*. In order to generate values of ξ, the medication analysis generation module 128 generates, based on the vector data 126, B, a p×k matrix of regression coefficients, and ϵ, a k×1 vector of residuals based on regression analysis. Equation 2 is an example of the system 100 that uses B and a k-factor model as the covariate effects on the common factors obtained by the factor generation module 118.

In some implementations, factors obtained by the factor generation module 118 are weighted by matrices applied to the factors For example, a matrix of factor loadings can be used, as shown in Equation 2, to influence the analysis of the completed assessment 116 based on the loaded factors. In some implementations, the factor loadings may be obtained according to a loading pattern. For example, a loading pattern may be configured to fit the data corresponding to the factors of the factor generation module 118.

In some implementations, a three-stage procedure may be used to estimate the factor loadings and recover a sparse factor loading pattern. For example, stage 1 can include estimating a matrix, such as a polychoric correlation matrix. Stage 2 can include obtaining a maximum likelihood estimate of the factor loadings matrix. For example, the maximum likelihood can include evaluating a function corresponding to a minimum for a series of inputs corresponding to the polychoric correlation matrix, such as argmin [log|Σ(Λ)|+tr{Σ(Λ)⁻¹ \hat{R}}]. Stage 2 may also include factorization of a matrix, such as the polychoric correlation matrix. Stage 3 can include constructing a log-likelihood function to obtain a regularized solution, where the regularized solution corresponds to the factor loadings. For example, the log-likelihood function can include F(λ)+P (∥λ∥₁;η) where F(λ)=log|Σ(λ)|+tr{Σ(λ)⁻¹\hat{R}} and P(·;η) is a specified penalty function with a tuning parameter η. A regularized solution may take the form of \hat{λ}(\hat{η})=argmin {F(λ)+P (∥λ∥₁; η)}. In some implementations, the optimal one \hat{η}, is chosen based on functions corresponding to one or more criterions, such as the Akaike information criterion or the Bayesian information criterion.

The three-stage process can result in a number of improvements over existing solutions known in the art. First, when the dimension of the ordinal outcome is high, which may be the case using a PANSS medical scale for schizophrenia studies, separate stages of computation performed by one or more components of the system 100, such as the three-stage procedure discussed above, can greatly reduce the computation burden of a system, such as the system 100, since a large portion of the unknown parameters come from the penalization-free thresholds that determine the ordinal manifest variables. Second, when incorporating the regularization, the loading factor matrix can be efficiently calculated with user-specified penalty related parameters. In some cases, the penalty related parameters can be programmed into the system 100 by a component of the system 100, such as the second user device 132. In addition, the three-stage approach can be optimized for a machine learning model hosted by one or more components of the system 100 as shown in FIG. 1 to generate a factor loading matrix as an output of a machine learning model trained using the processes and outputs corresponding to the three-stage process.

In some implementations, the medical analysis generation module 128 uses other models based on one or more other data sources. For example, the medical analysis generation module 128 can receive input based on data structures representing the completed assessment 116. In some cases, one or more items of the completed assessment 116 are used to update the joint model used by the medical analysis generation module 128. In some implementations, the medical analysis generation module 128 receives input directly from the factor generation module 118, the covariate identification module 120, and the ordinal extraction module 122. The data from the factor generation module 118, the covariate identification module 120, and the ordinal extraction module 122 can be stored in one or more fields corresponding to the completed assessment 116 and the first user 102. In some implementations, the joint model defined in part by Equation 2 represents but one of a plurality of possible alternative models available to the medical analysis generation module 128. In some cases, the medical analysis generation module 128 chooses from among the plurality of alternative models based on input data such as the completed assessment or other data corresponding to the system 100. The medical analysis generation module 128 then generates output data based on the chosen model.

The example joint model discussed above in Equation 2, in reference to the example of FIG. 1, may be rewritten compactly as:

y*=ΛB′x+Λϵ+δ (3)

Depending on the implementation, the medical analysis generation module 128 processes one or more computations based on the given joint model. For example, considering the joint model defined, in part, by Equation 3, the medical analysis generation module 128 computes one or more values corresponding to the joint model and a derived likelihood contribution:

∫_{α}_{_{q}}_{Y}_{_{q}}₋₁^{α}^{_{q}}^{Y}^{_{q}} . . . ∫_{α}_{₁}_{Y}_{₁}₋₁^{α}^{₁}^{Y}^{₁}ƒ_{q}(y*;ΛB′x,Σ)dy* (4)

In the example of Equation 4, the function ƒ_{q}(; μ, Σ) is a q-dimensional normal density function with mean μ and correlation matrix Σ. In some cases, the likelihood function corresponding to the likelihood contribution can be expressed, for n observations, such as n patients where each patient is similar to the first user 102 in that each patient provides data for a system such as the system 100, can be expressed as:

Lₙ(θ)=Πᵢ₌₁ⁿ∫_{α}_{_{q}}_{Y}_{_{iq}}₋₁^{α}^{_{q}}^{Y}^{_{q}} . . . ∫_{α}_{₁}_{Y}_{ᵢ₁}₋₁^{α}^{₁}^{Y}^{ᵢ₁}ƒ_{q}(y*;ΛB′x,Σ)dy* (5)

Computations corresponding to a chosen joint model, such as computations described in Equation 5, are computed by the medical analysis generation module 128. In some cases, approximation techniques are used to compute estimations of Equation 5. For example, in some cases, the medical analysis generation module 128 can compute a pairwise log-likelihood function based on Equation 5. The pairwise log-likelihood function can, in some cases, reduce the computation complexity of medical analysis output generated by the medical analysis generation module 128. For example, in some cases, a pairwise log-likelihood function can be expressed as:

plₙ(θ)=Σᵢ₌₁ⁿΣ_{1≤j<l≤q} log{∫_{α}_{ₗ}_{Y}_{ᵢₗ}₋₁^{α}^{ₗ}^{Y}^{ᵢₗ}∫_{α}_{ⱼ}_{Y}_{ᵢⱼ}₋₁^{α}^{ⱼ}^{Y}^{ᵢⱼ}ƒ₂(yⱼ*,yₗ*,μᵢⱼₗ,Σᵢₗ)dyⱼ*dyₗ*} (6)

In Equation 6, μᵢⱼₗ can be defined by ΛB′x and ƒ₂(_, _; μᵢⱼₗ, Σⱼₗ), similar to the function ƒ_{q}(; μ,Σ) of Equation 4, and is a bivariate normal density function with mean μᵢⱼₗ and covariance matrix Σⱼₗ. The pairwise log-likelihood function described in Equation 6 is a special case of a composite likelihood function. The medical analysis generation module 128 can use a function, such as the function described in Equation 6, to compute values based on the vector data 126 or other data of the system 100. In some cases, the medical analysis generation module 128 obtains additional data based on other users, or other patients, and uses the other data within an equation, such as the Equation 6, to compute an efficacy estimation for a psychiatric assessment.

The medical analysis generation module 128 computes one or more values and packages the one or more values in a data packet 130. The first computer 108 obtains the data packet 130 which includes one or more medical analyses based on the one or more values computed by the medical analysis generation module 128. In the example of FIG. 1, the first computer 108 sends the first data packet 130 to the second user device 132 over the communication network 107. In some cases, a request from the second user device 132 is received prior to the first computer 108 sending the first data packet to the second user device 132. The data packet 130 is configured to, when received by the second user device 132, enable operations by the second user device 132 to display one or more elements related to the data packet 130 and the one or more medical analyses. The second user device 132 can use a screen or be connected to another form of display to show the second user 134 the one or more medical analyses.

In some implementations, the medical analyses include efficacy estimations of psychiatric treatment. For example, based on the responses recorded in the completed assessment 116, and the data extracted by the factor generation module 118, the covariate identification module 120, the ordinal extraction module 122, and the vector generation module 124, the medical analysis generation module 128 can compute one or more values indicating whether or not a treatment is effective for the first user 102 based on one or more data items of a trial. In some cases, the one or more data items of a trial include one or more other completed assessments from other users undergoing treatment for a given condition such as a psychiatric condition.

In some implementations, the system 100 of FIG. 1 may be configured to process any multivariate ordinal data. For example, the first user 102 can send a request for user feedback to the first computer 108 from the first user device 104. In some implementations, communication to and from the first computer 108 may be encrypted. For example, the completed assessment 116 can be encrypted using a private key. In some cases, the private key can be associated with the first user device 104 or the first user 102. The first computer 108 can then decrypt the completed assessment 116 using a public key, such as a public key corresponding to the first user device 104 or the first user 102. In this way, user specific data included in the completed assessment 116 can be protected. In addition, in some implementations, the encryption can be further used to determine the origin of the completed assessment 116, such as by sequentially applying a decryption algorithm with one or more obtained public keys to the encrypted completed assessment 116.

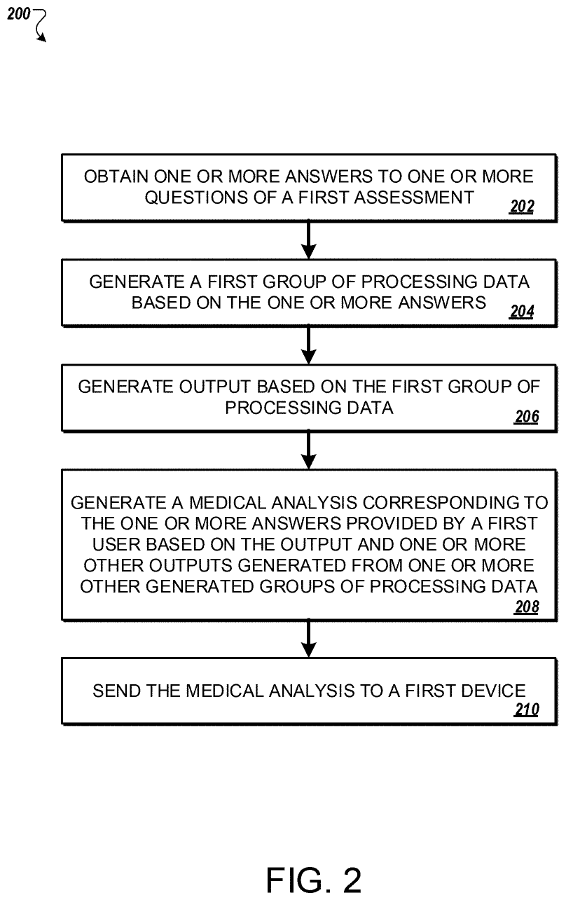

FIG. 2 is a flowchart for an example of a process 200 for generating medical analysis using a joint model based on multivariate ordinal data. The process 200 may be performed by one or more electronic systems, for example, the system 100 of FIG.

The process 200 includes obtaining one or more answers to one or more questions of a first assessment (202). For example, the first computer 108 obtains the completed assessment 116 in stage A of FIG. 1.

The process 200 includes generating a first group of processing data based on the one or more answers (204). For example, as shown in FIG. 1, the factor generation module 118, the covariate identification module 120, and the ordinal extraction module 122 generate processing data and send corresponding processing data to the vector generation module 124.

The process 200 includes generating output based on the first group of processing data (206). For example, the vector generation module 124 of FIG. 1 receives the processing data based on the output of the factor generation module 118, the covariate identification module 120, and the ordinal extraction module 122 and generates the vector data 126 based on the completed assessment 116 and the output of the covariate identification module 120, and the ordinal extraction module 122. The vector data 126 includes factors obtained by the factor generation module 118, covariates obtained by the covariate identification module 120, and ordinal values obtained by the ordinal extraction module 122.

The process 200 includes generating a medical analysis corresponding to the one or more answers provided by a first user based on the output and one or more other outputs generated from one or more other generated groups of processing data (208). For example, the medication analysis generation module 128 of FIG. 1 computes one or more values and generates the data packet 130 based on one or more values corresponding to one or more computations.

The process 200 includes sending the medical analysis to a first device (210). For example, in FIG. 1, the first computer 108 sends the first data packet 130 to the second user device 132 over the communication network 107. The data packet 130 is configured to, when received by the second user device 132, enable operations by the second user device 132 to display one or more elements related to the data packet 130 and the one or more medical analyses. The second user device 132 can use a screen or be connected to another form of display to show the second user 134 the one or more medical analyses.

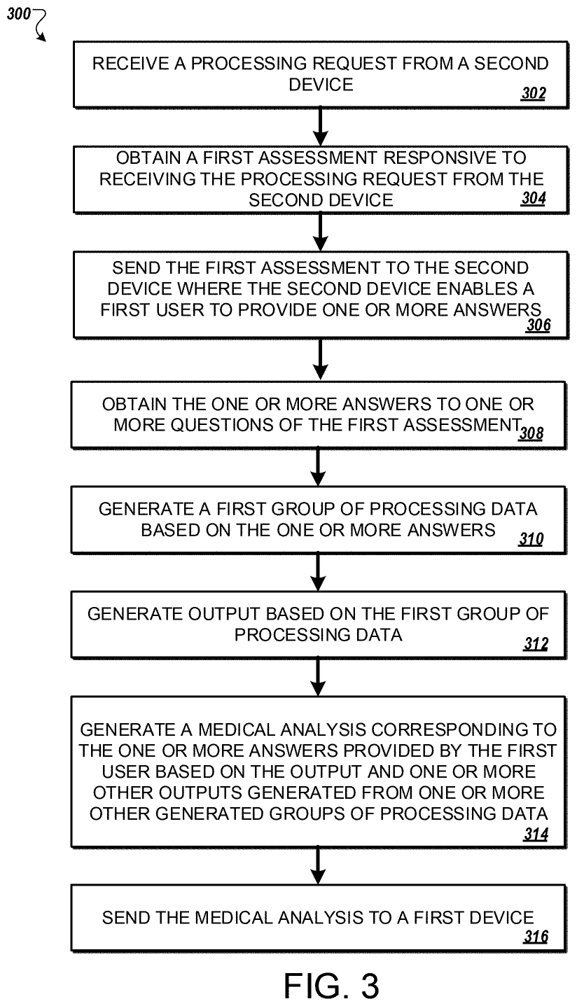

FIG. 3 is a flowchart for an example of a process 300 for establishing a psychiatric assessment and generating medical analysis using a joint model based on multivariate ordinal data. The process 300 may be performed by one or more electronic systems, for example, the system 100 of FIG. 1.

The process 300 includes receiving a processing request from a second device (302). For example, as shown in FIG. 1, the first user 102 uses the first user device 104 to send an assessment request 106 to the first computer 108. The assessment request 106 includes details related to the first user 102 and one or more assessments relevant to the first user 102. The assessment request 106 is sent over the communication network 107 to the first computer 108.

The process 300 includes obtaining a first assessment responsive to receiving the processing request from the second device (304). For example, as shown in FIG. 1, the first computer 108 receives the assessment request 106 and retrieves a corresponding assessment 114, based on the input of the assessment request 106. The first computer 108 obtains the assessment 114 from the one or more assessments stored on the assessment database 112.

The process 300 includes sending the first assessment to the second device where the second device enables a first user to provide one or more answers (306). For example, as shown in FIG. 1, the first computer 108 sends the assessment 114 to the first user device 104 where the first computer 108 and the first user device 104 are communicably connected by the communication network 107.

The process 300 includes obtaining the one or more answers to one or more questions of the first assessment (308). For example, the first computer 108 obtains the completed assessment 116 in stage A of FIG. 1.

The process 300 includes generating a first group of processing data based on the one or more answers (310). For example, as shown in FIG. 1, the factor generation module 118, the covariate identification module 120, and the ordinal extraction module 122 generate processing data and send corresponding processing data to the vector generation module 124.

The process 300 includes generating output based on the first group of processing data (312). For example, the vector generation module 124 of FIG. 1 receives the processing data based on the output of the factor generation module 118, the covariate identification module 120, and the ordinal extraction module 122 and generates the vector data 126 based on the completed assessment 116 and the output of the covariate identification module 120, and the ordinal extraction module 122. The vector data 126 includes factors obtained by the factor generation module 118, covariates obtained by the covariate identification module 120, and ordinal values obtained by the ordinal extraction module 122.

The process 300 includes generating a medical analysis corresponding to the one or more answers provided by the first user based on the output and one or more other outputs generated from one or more other generated groups of processing data (314). For example, the medication analysis generation module 128 of FIG. 1 computes one or more values and generates the data packet 130 based on one or more values corresponding to one or more computations.

The process 300 includes sending the medical analysis to a first device (316). For example, in FIG. 1, the first computer 108 sends the first data packet 130 to the second user device 132 over the communication network 107. The data packet 130 is configured to, when received by the second user device 132, enable operations by the second user device 132 to display one or more elements related to the data packet 130 and the one or more medical analyses. The second user device 132 can use a screen or be connected to another form of display to show the second user 134 the one or more medical analyses.

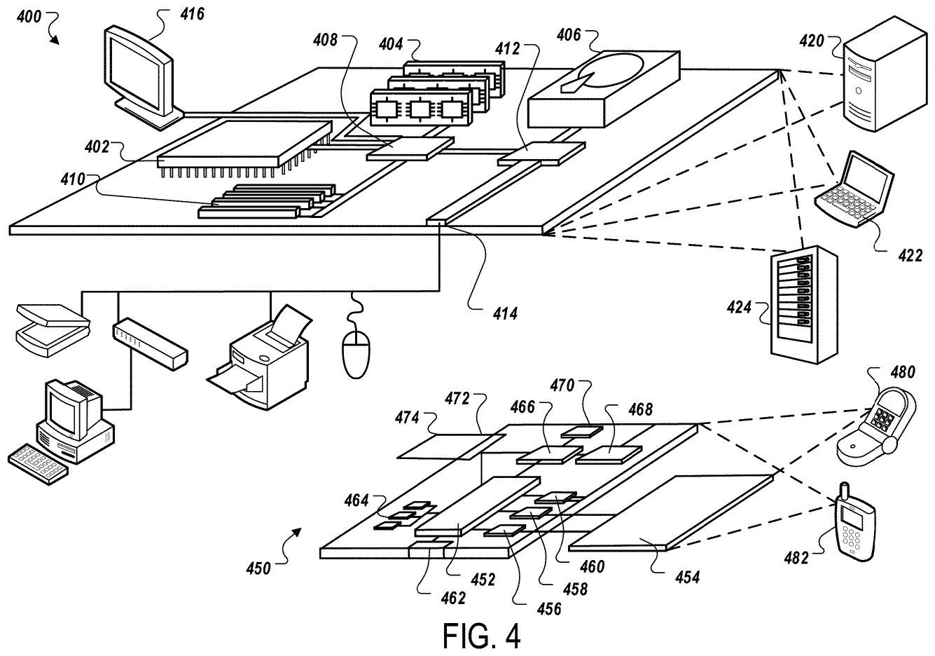

FIG. 4 is a diagram of computer system components that can be used to implement a system for generating medical analysis using a joint model based on multivariate ordinal data. The computing system includes computing device 400 and a mobile computing device 450 that can be used to implement the techniques described herein. For example, one or more components of the system 100 could be an example of the computing device 400 or the mobile computing device 450, such as a computer system implementing any one of the multiple components shown in FIG. 1

The computing device 400 is intended to represent various forms of digital computers, such as laptops, desktops, workstations, personal digital assistants, servers, blade servers, mainframes, and other appropriate computers. The mobile computing device 450 is intended to represent various forms of mobile devices, such as personal digital assistants, cellular telephones, smart-phones, mobile embedded radio systems, radio diagnostic computing devices, and other similar computing devices. The components shown here, their connections and relationships, and their functions, are meant to be examples only, and are not meant to be limiting.

The computing device 400 includes a processor 402, a memory 404, a storage device 406, a high-speed interface 408 connecting to the memory 404 and multiple high-speed expansion ports 410, and a low-speed interface 412 connecting to a low-speed expansion port 414 and the storage device 406. Each of the processor 402, the memory 404, the storage device 406, the high-speed interface 408, the high-speed expansion ports 410, and the low-speed interface 412, are interconnected using various busses, and may be mounted on a common motherboard or in other manners as appropriate. The processor 402 can process instructions for execution within the computing device 400, including instructions stored in the memory 404 or on the storage device 406 to display graphical information for a GUI on an external input/output device, such as a display 416 coupled to the high-speed interface 408. In other implementations, multiple processors and/or multiple buses may be used, as appropriate, along with multiple memories and types of memory. In addition, multiple computing devices may be connected, with each device providing portions of the operations (e.g., as a server bank, a group of blade servers, or a multi-processor system). In some implementations, the processor 402 is a single threaded processor. In some implementations, the processor 402 is a multi-threaded processor. In some implementations, the processor 402 is a quantum computer.

The memory 404 stores information within the computing device 400. In some implementations, the memory 404 is a volatile memory unit or units. In some implementations, the memory 404 is a non-volatile memory unit or units. The memory 404 may also be another form of computer-readable medium, such as a magnetic or optical disk.

The storage device 406 is capable of providing mass storage for the computing device 400. In some implementations, the storage device 406 may be or include a computer-readable medium, such as a floppy disk device, a hard disk device, an optical disk device, or a tape device, a flash memory or other similar solid-state memory device, or an array of devices, including devices in a storage area network or other configurations. Instructions can be stored in an information carrier. The instructions, when executed by one or more processing devices (for example, processor 402), perform one or more methods, such as those described above. The instructions can also be stored by one or more storage devices such as computer- or machine readable mediums (for example, the memory 404, the storage device 406, or memory on the processor 402). The high-speed interface 408 manages bandwidth-intensive operations for the computing device 400, while the low-speed interface 412 manages lower bandwidth-intensive operations. Such allocation of functions is an example only. In some implementations, the high speed interface 408 is coupled to the memory 404, the display 416 (e.g., through a graphics processor or accelerator), and to the high-speed expansion ports 410, which may accept various expansion cards (not shown). In the implementation, the low-speed interface 412 is coupled to the storage device 406 and the low-speed expansion port 414. The low-speed expansion port 414, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 400 may be implemented in a number of different forms, as shown in the figure. For example, it may be implemented as a standard server 420, or multiple times in a group of such servers. In addition, it may be implemented in a personal computer such as a laptop computer 422. It may also be implemented as part of a rack server system 424. Alternatively, components from the computing device 400 may be combined with other components in a mobile device, such as a mobile computing device 450. Each of such devices may include one or more of the computing device 400 and the mobile computing device 450, and an entire system may be made up of multiple computing devices communicating with each other.

The mobile computing device 450 includes a processor 452, a memory 464, an input/output device such as a display 454, a communication interface 466, and a transceiver 468, among other components. The mobile computing device 450 may also be provided with a storage device, such as a micro-drive or other device, to provide additional storage. Each of the processor 452, the memory 464, the display 454, the communication interface 466, and the transceiver 468, are interconnected using various buses, and several of the components may be mounted on a common motherboard or in other manners as appropriate.

The processor 452 can execute instructions within the mobile computing device 450, including instructions stored in the memory 464. The processor 452 may be implemented as a chipset of chips that include separate and multiple analog and digital processors. The processor 452 may provide, for example, for coordination of the other components of the mobile computing device 450, such as control of user interfaces, applications run by the mobile computing device 450, and wireless communication by the mobile computing device 450.

The processor 452 may communicate with a user through a control interface 458 and a display interface 456 coupled to the display 454. The display 454 may be, for example, a TFT (Thin-Film-Transistor Liquid Crystal Display) display or an OLED (Organic Light Emitting Diode) display, or other appropriate display technology. The display interface 456 may include appropriate circuitry for driving the display 454 to present graphical and other information to a user. The control interface 458 may receive commands from a user and convert them for submission to the processor 452. In addition, an external interface 462 may provide communication with the processor 452, so as to enable near area communication of the mobile computing device 450 with other devices. The external interface 462 may provide, for example, for wired communication in some implementations, or for wireless communication in other implementations, and multiple interfaces may also be used.

The memory 464 stores information within the mobile computing device 450. The memory 464 can be implemented as one or more of a computer-readable medium or media, a volatile memory unit or units, or a non-volatile memory unit or units. An expansion memory 474 may also be provided and connected to the mobile computing device 450 through an expansion interface 472, which may include, for example, a SIMM (Single In Line Memory Module) card interface. The expansion memory 474 may provide extra storage space for the mobile computing device 450, or may also store applications or other information for the mobile computing device 450. Specifically, the expansion memory 474 may include instructions to carry out or supplement the processes described above, and may include secure information also. Thus, for example, the expansion memory 474 may be provide as a security module for the mobile computing device 450, and may be programmed with instructions that permit secure use of the mobile computing device 450. In addition, secure applications may be provided via the SIMM cards, along with additional information, such as placing identifying information on the SIMM card in a non-hackable manner.

The memory may include, for example, flash memory and/or NVRAM memory (nonvolatile random access memory), as discussed below. In some implementations, instructions are stored in an information carrier such that the instructions, when executed by one or more processing devices (for example, processor 452), perform one or more methods, such as those described above. The instructions can also be stored by one or more storage devices, such as one or more computer- or machine-readable mediums (for example, the memory 464, the expansion memory 474, or memory on the processor 452). In some implementations, the instructions can be received in a propagated signal, for example, over the transceiver 468 or the external interface 462.

The mobile computing device 450 may communicate wirelessly through the communication interface 466, which may include digital signal processing circuitry in some cases. The communication interface 466 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), LTE, 5G/6G cellular, among others. Such communication may occur, for example, through the transceiver 468 using a radio frequency. In addition, short-range communication may occur, such as using a Bluetooth, Wi-Fi, or other such transceiver (not shown). In addition, a GPS (Global Positioning System) receiver module 470 may provide additional navigation- and location-related wireless data to the mobile computing device 450, which may be used as appropriate by applications running on the mobile computing device 450.

The mobile computing device 450 may also communicate audibly using an audio codec 460, which may receive spoken information from a user and convert it to usable digital information. The audio codec 460 may likewise generate audible sound for a user, such as through a speaker, e.g., in a handset of the mobile computing device 450. Such sound may include sound from voice telephone calls, may include recorded sound (e.g., voice messages, music files, among others) and may also include sound generated by applications operating on the mobile computing device 450.

The mobile computing device 450 may be implemented in a number of different forms, as shown in the figure. For example, it may be implemented as a cellular telephone 480. It may also be implemented as part of a smart-phone 482, personal digital assistant, or other similar mobile device.

A number of implementations have been described. Nevertheless, it will be understood that various modifications may be made without departing from the spirit and scope of the disclosure. For example, various forms of the flows shown above may be used, with steps re-ordered, added, or removed.

Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, e.g., one or more modules of computer program instructions encoded on a computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the invention can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the invention can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the invention, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

Particular embodiments of the invention have been described. Other embodiments are within the scope of the following claims. For example, the steps recited in the claims can be performed in a different order and still achieve desirable results.

What is claimed is:

1. A computer-implemented method for analyzing multi-variate ordinal outcomes, the method comprising:
obtaining, by one or more computers, one or more answers to one or more questions of a first assessment;
generating, by the one or more computers, a first group of processing data based on the one or more answers, wherein the first group of processing data comprises one or more ordinal values, one or more covariates, and one or more factors;
generating, by the one or more computers, output data based on the first group of processing data;
generating, by the one or more computers, a medical analysis corresponding to the one or more answers provided by a first user based on the output data and one or more other outputs from one or more other generated groups of processing data; and
sending, by the one or more computers, the medical analysis to a first device.

2. The computer-implemented method of claim 1, further comprising:
receiving, by the one or more computers, a processing request from a second device;
responsive to receiving the processing request from the second device, obtaining, by the one or more computers, the first assessment, wherein the first assessment comprises the one or more questions, wherein the first assessment is stored within an assessment database of one or more assessments, and wherein the assessment database is communicably connected to the one or more computers; and
sending, by the one or more computers, the first assessment to the second device, wherein the second device enables the first user to provide the one or more answers to the one or more questions of the first assessment.

3. The computer-implemented method of claim 1, wherein generating the first group of processing data comprises:
extracting, by the one or more computers, the one or more ordinal values from the one or more answers;
identifying, by the one or more computers, the one or more covariates from the one or more answers; and
generating, by the one or more computers, the one or more factors based on the one or more answers.

4. The computer-implemented method of claim 1, wherein the one or more factors comprise one or more of negative symptoms, positive symptoms, disorganized thought, uncontrolled hostility or excitement, or anxiety or depression.

5. The computer-implemented method of claim 1, wherein the one or more covariates comprise one or more of treatment group, age, or gender.

6. The computer-implemented method of claim 1, wherein the medical analysis comprises a determined effect of a drug treatment, wherein the first user is engaged in the drug treatment.

7. A system for analyzing multi-variate ordinal outcomes, the system comprising:
one or more computers; and
one or more memories storing instructions that, when executed by the one or more computers, cause the one or more computers to perform operations, the operations comprising:
obtaining, by the one or more computers, one or more answers to one or more questions of a first assessment;
generating, by the one or more computers, a first group of processing data based on the one or more answers, wherein the first group of processing data comprises one or more ordinal values, one or more covariates, and one or more factors;
generating, by the one or more computers, output data based on the first group of processing data;
generating, by the one or more computers, a medical analysis corresponding to the one or more answers provided by a first user based on the output data and one or more other outputs from one or more other generated groups of processing data; and
sending, by the one or more computers, the medical analysis to a first device.

8. The system of claim 7, the operations further comprising:
receiving, by the one or more computers, a processing request from a second device;
responsive to receiving the processing request from the second device, obtaining, by the one or more computers, the first assessment, wherein the first assessment comprises the one or more questions, wherein the first assessment is stored within an assessment database of one or more assessments, and wherein the assessment database is communicably connected to the one or more computers; and
sending, by the one or more computers, the first assessment to the second device, wherein the second device enables the first user to provide the one or more answers to the one or more questions of the first assessment.

9. The system of claim 7, wherein generating the first group of processing data comprises:
extracting, by the one or more computers, the one or more ordinal values from the one or more answers;
identifying, by the one or more computers, the one or more covariates from the one or more answers; and
generating, by the one or more computers, the one or more factors based on the one or more answers.

10. The system of claim 7, wherein the one or more factors comprise one or more of negative symptoms, positive symptoms, disorganized thought, uncontrolled hostility or excitement, or anxiety or depression.

11. The system of claim 7, wherein the one or more covariates comprise one or more of treatment group, age, or gender.

12. The system of claim 7, wherein the medical analysis comprises a determined effect of a drug treatment, wherein the first user is engaged in the drug treatment.

13. A computer-readable storage medium storing instructions that, when executed by one or more computers, cause the one or more computers to perform operations, the operations comprising:
obtaining one or more answers to one or more questions of a first assessment;
generating a first group of processing data based on the one or more answers, wherein the first group of processing data comprises one or more ordinal values, one or more covariates, and one or more factors;
generating output data based on the first group of processing data;
generating a medical analysis corresponding to the one or more answers provided by a first user based on the output data and one or more other outputs from one or more other generated groups of processing data; and
sending the medical analysis to a first device.

14. The computer-readable storage medium of claim 13, the operations further comprising:
receiving a processing request from a second device;
responsive to receiving the processing request from the second device, obtaining the first assessment, wherein the first assessment comprises the one or more questions, wherein the first assessment is stored within an assessment database of one or more assessments, and wherein the assessment database is communicably connected to the one or more computers; and
sending the first assessment to the second device, wherein the second device enables the first user to provide the one or more answers to the one or more questions of the first assessment.

15. The computer-readable storage medium of claim 13, wherein generating the first group of processing data comprises:
extracting, by the one or more computers, the one or more ordinal values from the one or more answers;
identifying, by the one or more computers, the one or more covariates from the one or more answers; and
generating, by the one or more computers, the one or more factors based on the one or more answers.

16. The computer-readable storage medium of claim 13, wherein the one or more factors comprise one or more of negative symptoms, positive symptoms, disorganized thought, uncontrolled hostility or excitement, or anxiety or depression.

17. The computer-readable storage medium of claim 13, wherein the one or more covariates comprise one or more of treatment group, age, or gender.

18. The computer-readable storage medium of claim 13, wherein the medical analysis comprises a determined effect of a drug treatment, wherein the first user is engaged in the drug treatment.
